(19) United States
(12) Patent Application Publication
(10) Pub. No.: US 2022/0259388 A1
(43) Pub. Date: Aug. 18, 2022

(54) PROCESS FOR CROSSLINKING A POLYMER

(71) Applicant: LABORATOIRES VIVACY, Paris (FR)

(72) Inventor: Guillaume BARRAL, Gruffy (FR)

(73) Assignee: LABORATOIRES VIVACY, Paris (FR)

(21) Appl. No.: 17/604,587

(22) PCT Filed: Apr. 17, 2020
(86) PCT No.: PCT/EP2020/060932

§ 371 Date: May 5, 2022

(30) Foreign Application Priority Data
Apr. 17, 2019 (FR) 19/04134

(51) Int. Cl. C08J 3/24; A61K 8/04; A61K 8/73; A61Q 19/08

(52) U.S. Cl. CPC C08J 3/247; A61K 8/042; A61K 8/735; A61K 2800/95; C08J 2305/08; A61K 2800/594; A61Q 19/08

(57) ABSTRACT

A process for crosslinking a polymer, includes at least the following steps: a) providing a polymer; b) providing a crosslinking agent; c) carrying out one or more crosslinking steps in the presence of the polymer and the crosslinking agent; d) obtaining a crosslinked polymer; wherein the crosslinking step or each of the crosslinking steps is carried out at constant temperature or at a temperature that varies linearly or in a stepwise manner, the constant or variable temperature being less than or equal to 15° C., and in that the crosslinking step c) has a duration of between 3 and 72 hours.

The invention relates to the field of polymer-based formulations used as biomaterials, and more particularly in the medical and esthetic fields. In all of these applications, the formulations must have optimized rheological properties and must guarantee good injectability and good in vivo performance.

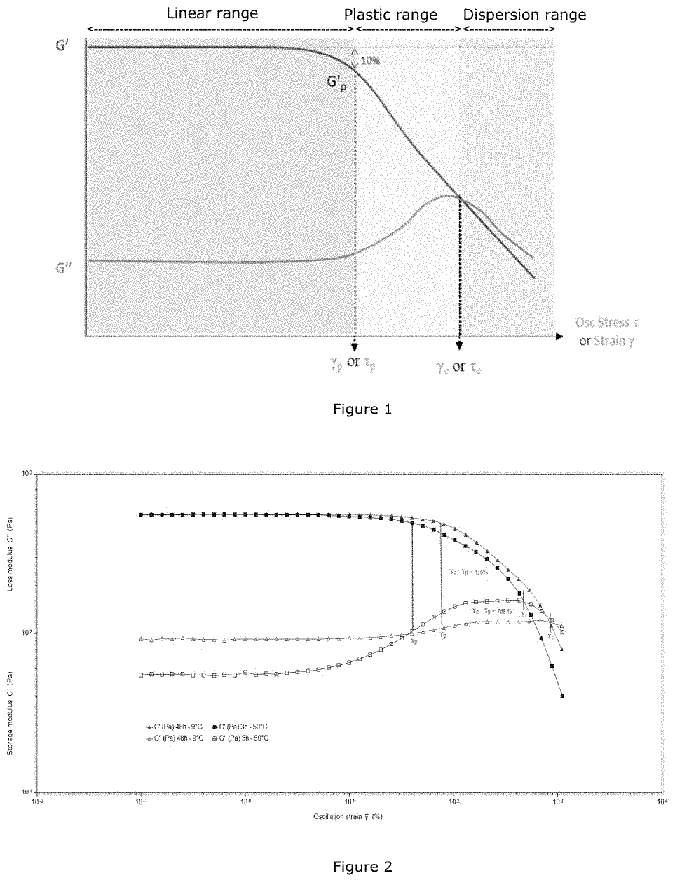

There is a need for products offering optimal characteristics and significant advantages in an esthetic application, with good damping capacity imparted by an optimized Tan (Tn δ) and good persistence in the injection zone imparted by a very wide plastic range. Surprisingly, it was demonstrated that such properties could be obtained using the crosslinking process under particular temperature and time conditions.
These properties, as illustrated in FIG. 1, can be obtained by optimizing the damping factor or tangent of the phase angle (Tan Δ (Tn δ)) while maintaining adequate rigidity (elastic modulus G′) and increasing the plastic range.

The plastic range is characterized by the evolution of the elastic modulus G′ and of the viscous modulus G″ as a function of the deformation applied to the product.

When the deformation applied to the gel is within the plastic range, it deforms the product without breaking it. When the yield point is exceeded (G′/G″ crossover), the product yields and the damping and filling properties of the product are no longer optimal.

FIG. 1 describes the series of ranges observed during the oscillation strain sweep.

It is generally observed that the more rigid (high G′) a product is, the smaller its plastic range is. In essence, a rigid product will generally be more brittle and less capable of being deformed.

Surprisingly, the process of the invention makes it possible to obtain a crosslinked product that is more rigid, but also capable of withstanding very high levels of deformation with the quality of a very wide plastic range.

The product obtained therefore has optimal characteristics in an esthetic application, where its good damping capacity imparted by the Tan Δ (Tn δ) and its good persistence in the injection zone imparted by a highly optimized plastic range enable it to offer significant advantages.

The invention relates to a process for preparing a crosslinked polymer-based formulation, for example a crosslinked hyaluronic acid-based formulation, and more particularly a crosslinking process that makes it possible to obtain particular properties, and specifically an optimized Tan Δ (Tn δ) and a wide plastic range t.

In the context of the present application, “crosslinking” is understood to mean the creation of covalent bonds between monomers of polymers.

When the crosslinking is achieved by means of a crosslinking agent, the crosslinking rate (X) can be calculated theoretically using the following formula:

$$X = \frac{\begin{matrix}number of moles of crosslinking \\ agent inroduced into the reaction medium\end{matrix}}{\begin{matrix}number of moles of repeating units ( disaccharide unit ) \\ introduced into the reaction medium\end{matrix}}$$

Thus, for example, if a medium comprises 100 disaccharide units, and said medium also comprises 10 molecules of crosslinking agent, then the crosslinking rate (X) will be as follows: X=10/100=0.1. This crosslinking rate is therefore not influenced by the degree of polymerization, by the molecular mass of the polymer chosen, or by the proportion of crosslinking agent that actually reacts with at least one function of the polymer. It is a theoretical determination taking into account only the quantities of crosslinking agent and repeating units placed in contact.

The crosslinking can also be evaluated a posteriori (after crosslinking), by means of the degree of modification (Mod). The Mod, unlike the crosslinking rate X, takes into account the proportion of crosslinking agent that actually reacts with at least one function of the polymer.

The degree of modification can be expressed as follows:

$$Mod ( \% ) = \frac{\begin{matrix}number of moles of crosslinking agent linked by at \\ least one covalent bond to at least one disaccharide unit\end{matrix}}{\begin{matrix}number of moles of repeating units \\ present in the reaction medium\end{matrix}} * 100$$

The repeating unit (or monomer), when the polymer is hyaluronic acid, is a disaccharide unit.

The determination of the values of the numerator and denominator depends on the polymer chosen and the crosslinking agent chosen, and are well known to the person skilled in the art. For example, in the particular case of a hyaluronic acid-based formulation crosslinked with BDDE, the process described in the publication L. Nord, A. Emi/son, C. Sturesson, A. H. Kenne, Degree of Modification of Hyaluronic Acid Dermal Fillers, 18th Congress of the EADV, Berlin, 2009 can be used.

In the particular case of a hyaluronic acid-based formulation crosslinked with BDDE, the degree of modification can be expressed as follows:

$$Mod ( \% ) = \frac{\begin{matrix}number of moles of BDDE linked by at least one covalent \\ bond to at least one dissacharide unit of hyaluronic acid\end{matrix}}{\begin{matrix}\begin{matrix}number of moles of repeating units \\ ( disaccharide unit of hyaluronic acid )\end{matrix} \\ present in the reaction medium\end{matrix}} * 100$$

For example, a hyaluronic acid-based formulation crosslinked with BDDE having a Mod of 1% indicates that it has one molecule of (single- or double-bonded) BDDE per 100 disaccharide units.

Traditionally, the crosslinking step is carried out at a temperature much higher than the ambient temperature, for a relatively short time.

Thus, for example in Example 1 of the application WO2009071697 in the name of the applicant, the crosslinking conditions are as follows: 50° C. for two hours and twenty minutes (2:20). These crosslinking conditions are fairly conventional and are applied almost systematically.

It is only quite recently that it has been proposed to use crosslinking temperatures lower than the conventionally used temperatures.

The application CN108774330 in the name of BLOOMAGE FREDA BIOPHARM CO LTD proposes, in the context of the preparation of a formulation intended to be applied to the skin, the implementation of a crosslinking at variable temperatures. More particularly, e.g. in example 2, a crosslinking is proposed in which the temperature is, successively, from 1 to 4° C., then 50° C., and the operation is repeated several times. The conclusions of Table 1 are that in the high-temperature stage, a temperature between 50° C. and 80° C. is ideal. It should be noted that the formulations disclosed are biphasic formulations, for external use (no injection, only application to the skin), and that no mention is made with respect to the rheology of the formulations and the quantification of the crosslinking performed. Lastly, in this application, it is not established that a crosslinking occurs between 1 and 4° C.

The application CN107936272 in the name of BLOOMAGE FREDA BIOPHARM CO LTD proposes a crosslinking process that also provides for an alternation of low (0-10° C.) and high (30-60° C.) temperatures. It should be noted that no mention is made with respect to the rheology of the formulations.

The application CN108774330 in the name of BLOOMAGE FREDA BIOPHARM CO LTD, proposes a crosslinking process that also provides for an alternation of low (1-4° C.) and high (50-80° C.) temperatures.

The application WO17016917 in the name of GALDERMA SA proposes a crosslinking performed with a high concentration of hydroxide ions (1.5-8%), a high concentration of hyaluronic acid (more than 10%), and very specific temperature and time conditions. For example, the process of example 3 corresponds to the following conditions: 29° C., 16 hours.

The application CN103146003 in the name of SHANGHAI QISHENG BIOLOG PREPARATION CO LTD discloses a crosslinking process that also comprises an alternation of low and high temperatures, for example in embodiment 1, the crosslinking starts at 4° C. and ends at 40° C. It should be noted that no mention is made with respect to the rheology of the formulations or the effect of the low temperature on the crosslinking reaction.

The application US2010/0261893 in the name of Tor-Chern Chen discloses examples of crosslinking at temperatures between 10 and 30° C., particularly in order to reduce the percentage of crosslinking agent comprising a free end after reaction. When the operating temperatures of the processes are lower than 20° C., the reaction times are always greater than 10 days and can be 28 days. Furthermore, no analysis is made with respect to the rheological properties, the only goal is to reduce the crosslinking agent content in the finished product.

The application KR1018666678 in the name of the University of Seoul illustrates an exemplary embodiment of a process for crosslinking at less than 20° C., with reaction times that are greater than 14 days

In summary, in the above-mentioned applications, either the crosslinking is fully or partially performed at a temperature higher than 30° C. for reaction times of less than one day or, when the crosslinking temperatures are lower than 20° C., the reaction times are extremely long. Moreover, the rheological properties, when they are described, do not correspond to those sought and obtained under the conditions according to the invention.

As indicated above, it was demonstrated by the applicant that polymer-based formulations having, in particular, very advantageous rheological properties, with good damping capacity imparted by an optimized Tan Δ (Tn δ) and good persistence in the injection zone imparted by a very wide plastic range, can be obtained by means of a crosslinking performed exclusively at low temperature (less than or equal to 15° C.) in times compatible with industrial production, for example between 3 and 72 hours.

For example, in the case of hyaluronic acid-based formulations, it was demonstrated that the use of a low crosslinking temperature enabled:
the obtainment of polymers having a degree of modification Mod (%) lower than that obtained at higher temperatures, wherein:
   the elastic modulus G′ is optimized and is always lower than 1000 Pa;
   the viscous modulus G″ is optimized (values of G″ substantially higher than those of the formulations according to the prior art);
   with;
reduced degradation of the polymer during crosslinking;
optimization of the value of Tan Δ (Tn δ) (the ratio of the viscous modulus G″ divided by the elastic modulus G′) in order to obtain an improved formulation better able to withstand the stresses linked to the deformation of the product so as to have a target value within the range of 0.25 to 1 (0.25≤Tan Δ (Tn δ))≤1)
good injectability characteristics;
reduced energy consumption (relative to a high temperature, or even relative to variable temperatures);
a process that is reliable, reproducible, and requires little human intervention;
a simplified process;
a crosslinking that is very efficient and enables minimal modification of the polymer so as to ensure better biocompatibility (good rheological properties obtained, with a fairly low Mod).

Even more surprisingly, it was demonstrated that when a formulation based on several polymers (these polymers possibly being, for example, hyaluronic acids) crosslinked at low temperature prior to their interpenetration by mixing, was prepared according to the process of the invention, the formulations obtained by means of the process of the invention had further improved properties.

For example, as will be demonstrated in the examples, the crosslinking process according to the invention makes it possible to obtain particularly advantageous values of Tan Δ 1 Hz (target values >0.25). It was observed that for values of Tan Δ >0.25, the material obtained showed a reduction in its brittle characteristic and an increase in its deformation capacity; which seems ideal for a medical filling application in which the damping of deformation is important. In the particular case of esthetics, this characteristic is a significant advantage because it enables a natural correction after injection. We thus seek in this process to optimize the damping factor while retaining a rigidity G′ that is satisfactory and equivalent to the prior art.

Finally, it was demonstrated that the efficiency of the crosslinking was very good since very good rheological properties are obtained with a relatively low Mod (%); this makes it possible to ensure improved biocompatibility.

The invention relates to a process for crosslinking a polymer, comprising at least the following steps:
a) providing a polymer;
b) providing a crosslinking agent;
c) carrying out one or more crosslinking step(s) in the presence of said polymer and said crosslinking agent;
d) obtaining a crosslinked polymer;
   characterized in that the crosslinking step or each of the crosslinking steps is carried out at a constant temperature or at a temperature that varies linearly or in a stepwise manner, said constant or variable temperature being less than or equal to 15° C. (temperature ≤15° C.).

The process for crosslinking a polymer according to the invention is also characterized in that the crosslinked polymer obtained in step d) has a G′≤1000 Pa.

In one embodiment, the process for crosslinking a polymer according to the invention is characterized in that the crosslinked polymer obtained in step d) has a G′≤800 Pa.

In one embodiment, the process for crosslinking a polymer according to the invention is characterized in that the crosslinked polymer obtained in step d) has a G′≤600 Pa.

In one embodiment, the process for crosslinking a polymer according to the invention is characterized in that the crosslinked polymer obtained in step d) has a G′≤500 Pa.

In one embodiment, the process for crosslinking a polymer according to the invention is characterized in that the crosslinked polymer obtained in step d) has a G′≤300 Pa.

In one embodiment, the process for crosslinking a polymer according to the invention is characterized in that the crosslinked polymer obtained in step d) has a G′≤200 Pa.

In one embodiment, the process for crosslinking a polymer according to the invention is characterized in that the crosslinked polymer obtained in step d) has a G′≤100 Pa.

In one embodiment, the process for crosslinking a polymer according to the invention is characterized in that the crosslinked polymer obtained in step d) has a G′≤50 Pa.

In one embodiment, the process for crosslinking a polymer according to the invention is characterized in that the crosslinked polymer obtained in step d) has a G′ having a value within the range of 50 to 610 Pa (50≤G′≤610).

The process for crosslinking a polymer according to the invention is also characterized in that the crosslinked polymer obtained in step d) has a Tan Δ (Tn δ) whose value is between 0.25 and 1 (0.25≤Tan Δ (Tn δ))≤1).

In one embodiment, the process for crosslinking a polymer according to the invention is characterized in that the crosslinked polymer obtained in step d) has a Tan Δ (Tn δ) whose value is between 0.50 and 1 (0.50≤Tan Δ (Tn δ))≤1).

In one embodiment, the process for crosslinking a polymer according to the invention is characterized in that the crosslinked polymer obtained in step d) has a Tan Δ (Tn δ) whose value is between 0.75 and 1 (0.75≤Tan Δ (Tn δ))≤1)

In one embodiment, the process for crosslinking a polymer according to the invention is characterized in that the crosslinked polymer obtained in step d) has a tan whose value is within the range of 0.3 to 0.6 (0.3≤Tan Δ (Tn δ)≤0.6).

The invention also relates to the polymer obtained by the process according to the invention.

In one embodiment, the polymer according to the invention has a G′≤1000 Pa and a tan Δ (Tn δ) whose value is between 0.25 and 1 (0.25≤Tan Δ (Tn δ))≤1)

In one embodiment, the polymer according to the invention has a G′≤800 Pa.

In one embodiment, the polymer according to the invention has a G′≤600 Pa.

In one embodiment, the polymer according to the invention has a G′≤500 Pa.

In one embodiment, the polymer according to the invention has a G′≤300 Pa.

In one embodiment, the polymer according to the invention has a G′≤200 Pa.

In one embodiment, the polymer according to the invention has a G′≤100 Pa.

In one embodiment, the polymer according to the invention has a G′≤50 Pa.

In one embodiment, the polymer according to the invention has a G′ having a value within the range of 50 to 610 Pa (50≤G′≤610).

In one embodiment, the polymer according to the invention has a tan Δ (Tn δ) whose value is between 0.50 and 1 (0.50≤tan Δ (Tn δ))≤1).

In one embodiment, the polymer according to the invention has a Tan Δ (Tn δ) whose value is between 0.75 and 1 (0.75≤Tan Δ (Tn δ))≥1)

In one embodiment, the polymer according to the invention has a Tan Δ (Tn δ) whose value is between 0.3 and 0.6 (0.3≤Tan Δ (Tn δ)≤0.6).

In one embodiment, the polymer according to the invention is characterized in that it is chosen from the group of polysaccharides.

In one embodiment, the polymer according to the invention is characterized in that it is comprised of a mixture of polymers.

In one embodiment, the polymer according to the invention is a mixture of hyaluronic acids or hyaluronic acid salts.

In one embodiment, the process for crosslinking a polymer according to the invention is characterized in that the crosslinking step or each of the crosslinking steps is carried out at a constant temperature.

In one embodiment, the process for crosslinking a polymer according to the invention is characterized in that at least one crosslinking step is carried out at a variable temperature.

In one embodiment, the process for crosslinking a polymer according to the invention is characterized in that at least one crosslinking step is carried out at a temperature that varies linearly.

In one embodiment, the process for crosslinking a polymer according to the invention is characterized in that at least one crosslinking step is carried out at a temperature that varies in a stepwise manner.

In one embodiment, the process for crosslinking a polymer according to the invention is characterized in that said constant or variable temperature is less than or equal to 15° C. (temperature≤15° C.).

In one embodiment, the process for crosslinking a polymer according to the invention is characterized in that at least 90% of the crosslinking is carried out at a constant or variable temperature less than or equal to 15° C. (temperature≤15° C.).

In one embodiment, the process for crosslinking a polymer according to the invention is characterized in that at least 80% of the crosslinking is carried out at a constant or variable temperature less than or equal to 15° C. (temperature≤15° C.).

In one embodiment, the process for crosslinking a polymer according to the invention is characterized in that at least 70% of the crosslinking is carried out at a constant or variable temperature less than or equal to 15° C. (temperature≤15° C.).

In one embodiment, the process for crosslinking a polymer according to the invention is characterized in that the crosslinking step at a constant or variable temperature less than or equal to 15° C. (temperature≤15° C.) represents at least 90% of the contact time of the reagents.

In one embodiment, the process for crosslinking a polymer according to the invention is characterized in that the crosslinking step at a constant or variable temperature less than or equal to 15° C. (temperature≤15° C.) represents at least 80% of the contact time of the reagents.

In one embodiment, the process for crosslinking a polymer according to the invention is characterized in that the crosslinking step at a constant or variable temperature less than or equal to 15° C. (temperature≤15° C.) represents at least 70% of the contact time of the reagents.

In one embodiment, the process for crosslinking a polymer according to the invention is characterized in that said constant or variable temperature is less than or equal to 12° C. (temperature≤12° C.).

In one embodiment, the process for crosslinking a polymer according to the invention is characterized in that said constant or variable temperature is less than or equal to 10° C. (temperature≤10° C.).

In one embodiment, the process for crosslinking a polymer according to the invention is characterized in that said constant or variable temperature is less than or equal to 9° C. (temperature≤9° C.).

The solidification temperature of the reaction medium is understood to mean the temperature at which the medium becomes solid. For an aqueous medium, this temperature will be 0° C., or slightly lower as a function of the concentration of salts in said medium.

In one embodiment, the process for crosslinking a polymer according to the invention is characterized in that said constant or variable temperature is between the solidification temperature and 15° C.

In one embodiment, the process for crosslinking a polymer according to the invention is characterized in that said constant or variable temperature is between the solidification temperature and 10° C.

In one embodiment, the process for crosslinking a polymer according to the invention is characterized in that said constant or variable temperature is between the solidification temperature and 9° C.

In one embodiment, the process for crosslinking a polymer according to the invention is characterized in that prior to step c), a step for dissolving said polymer is performed.

The dissolution of the polymer is performed by adding water or an aqueous saline solution, for example a phosphate buffer solution, for example PBS, or by adding a sodium hydroxide or acid solution so as to obtain the pH compatible with the implementation of the crosslinking reaction.

In one embodiment, the process for crosslinking a polymer according to the invention is characterized in that, in step c) at the latest, a step for adjusting the pH to a crosslinking pH is performed.

The adjustment of the pH is performed by adding preferably a mineral acid solution, for example hydrochloric acid, or preferably a mineral base, for example sodium hydroxide or potassium hydroxide, said acids and bases being added in a quantity that makes it possible to obtain the target crosslinking pH.

In one embodiment, the process for crosslinking a polymer according to the invention is characterized in that, in step c) at the latest, a step for adjusting the pH to a crosslinking pH suitable for said crosslinking agent is performed.

In one embodiment, the process for crosslinking a polymer according to the invention is characterized in that in step c) at the latest, a step for adjusting the pH to a crosslinking pH greater than 10 is performed.

In one embodiment, the process for crosslinking a polymer according to the invention is characterized in that in step c) at the latest, a step for adjusting the pH to a crosslinking pH less than 3 is performed.

In one embodiment, the process for crosslinking a polymer according to the invention is characterized in that in step c) at the latest, a step for adjusting the pH to a crosslinking pH greater than 10 is performed, said crosslinking agent being BDDE.

In one embodiment, the process for crosslinking a polymer according to the invention is characterized in that in step c) at the latest, a step for adjusting the pH to a crosslinking pH less than 3 is performed, said crosslinking agent being BDDE.

In one embodiment, the process for crosslinking a polymer according to the invention is characterized in that in step c) at the latest, a step for adjusting the pH to a crosslinking pH is performed, said crosslinking pH being greater than 10.

The crosslinking starts when the following 3 conditions are combined: the presence of the polymer, the presence of the crosslinking agent, and a reaction medium at an appropriate pH.

In one embodiment, the process for crosslinking a polymer according to the invention is characterized in that the crosslinking is initiated by the addition of said crosslinking agent.

In one embodiment, the process for crosslinking a polymer according to the invention is characterized in that the crosslinking is initiated by the addition of said polymer.

In one embodiment, the process for crosslinking a polymer according to the invention is characterized in that the crosslinking is initiated by the application of a crosslinking pH.

In one embodiment, the process for crosslinking a polymer according to the invention is characterized in that after step c), a step for adjusting the pH to a pH between 6 and 8 is performed.

In one embodiment, the process for crosslinking a polymer according to the invention is characterized in that after step c), a step for adjusting the pH to a pH between 6 and 8 is performed.

As a function of the pH of the reaction medium at the end of the crosslinking reaction, the adjustment of the pH is performed by adding a preferably mineral acid solution, for example hydrochloric acid, or a preferably mineral base, for example soda or lye, said acids and bases being added in a quantity that makes it possible to obtain a pH between 6 and 8.

In one embodiment, the process for crosslinking a polymer according to the invention is characterized in that after step c), a step for adjusting the pH to a pH between 6 and 8 is performed by adding at least one acid, i.e. hydrochloric acid (HCl).

In one embodiment, the process for crosslinking a polymer according to the invention is characterized in that prior to step d), a purification step is performed.

In one embodiment, the process for crosslinking a polymer according to the invention is characterized in that prior to step d), a step for purification by dialysis is performed.

In one embodiment, the process for crosslinking a polymer according to the invention is characterized in that prior to step d), a step for purification by dialysis is performed by means of a dialysis solution or solvent chosen from the group of phosphate buffers, for example PBS and water.

In one embodiment, the process for crosslinking a polymer according to the invention is characterized in that prior to step d), a step for eliminating said crosslinking agent is performed.

In one embodiment, the process for crosslinking a polymer according to the invention is characterized in that prior to step c), a step for cooling to the crosslinking temperature is performed.

In one embodiment, the process for crosslinking a polymer according to the invention is characterized in that said polymer of step a) is a mixture of polymers.

In the context of the present application, during step a), all of the polymers cited can be placed in contact in the form of a mixture with polymer of the same nature (for example a mixture of hyaluronic acid having various molecular masses) or of a different nature (for example a mixture of hyaluronic acid and chitosan). During the crosslinking step, it is possible to have co-crosslinking between the various polymers.

In one embodiment, the process for crosslinking a polymer according to the invention is characterized in that said polymer of step a) is a mixture of hyaluronic acids or hyaluronic acid salts.

In one embodiment, the process for crosslinking a polymer according to the invention is characterized in that said polymer of step a) is a mixture of 2 hyaluronic acids or hyaluronic acid salts.

In one embodiment, the process for crosslinking a polymer according to the invention is characterized in that said polymer of step a) is a mixture of 3 hyaluronic acids or hyaluronic acid salts.

In one embodiment, the process for crosslinking a polymer according to the invention is characterized in that said polymer of step a) is a mixture of 4 hyaluronic acids or hyaluronic acid salts.

In one embodiment, the process for crosslinking a polymer according to the invention is characterized in that the placement of said polymer in contact with the at least one crosslinking agent takes place in a solvent.

In one embodiment, the process for crosslinking a polymer according to the invention is characterized in that said at least one crosslinking agent is chosen from the group comprised of ethylene glycol diglycidyl ether, butanediol diglycidyl ether (BDDE), polyglycerol polyglycidyl ether, polyethylene glycol diglycidyl ether, polypropylene glycol diglycidyl ether, a bis- or polyepoxide such as 1,2,3,4-diepoxybutane or 1,2,7,8-diepoxyoctane, a dialkyl sulfone, divinyl sulfone, formaldehyde, epichlorohydrin or glutaraldehyde, carbodiimides such as for example 1-ethyl-3-[3-dimethylaminopropy]carbodiimide hydrochloride (EDC), trimetaphosphates such as for example sodium trimetaphosphate, calcium trimetaphosphate, or barium trimetaphosphate.

In one embodiment, the process for crosslinking a polymer according to the invention is characterized in that said at least one crosslinking agent is chosen from the group comprised of ethylene glycol diglycidyl ether, butanediol diglycidyl ether (BDDE), polyglycerol polyglycidyl ether, polyethylene glycol diglycidyl ether, polypropylene glycol diglycidyl ether, a bis- or polyepoxide such as 1,2,3,4-diepoxybutane or 1,2,7,8-diepoxyoctane, trimetaphosphates such as for example sodium trimetaphosphate, calcium trimetaphosphate, or barium trimetaphosphate.

In one embodiment, the process for crosslinking a polymer according to the invention is characterized in that said at least one crosslinking agent is chosen from the group comprised of ethylene glycol diglycidyl ether, butanediol diglycidyl ether (BDDE), polyglycerol polyglycidyl ether, polyethylene glycol diglycidyl ether, polypropylene glycol diglycidyl ether, a bis- or polyepoxide such as 1,2,3,4-diepoxybutane or 1,2,7,8-diepoxyoctane.

In one embodiment, the process for crosslinking a polymer according to the invention is characterized in that said at least one crosslinking agent is chosen from the group comprised of trimetaphosphates, such as for example sodium trimetaphosphate, calcium trimetaphosphate, or barium trimetaphosphate.

In one embodiment, the process for crosslinking a polymer according to the invention is characterized in that said at least one crosslinking agent is chosen from the group comprised of epoxides, for example 1,4-butanediol diglycidyl ether (BDDE), epihalohydrins, and divinyl sulfone (DVS).

In one embodiment, the process for crosslinking a polymer according to the invention is characterized in that said at least one crosslinking agent is divinyl sulfone (DVS).

In one embodiment, the process for crosslinking a polymer according to the invention is characterized in that said at least one crosslinking agent is 1,4-butanediol diglycidyl ether (BDDE).

In the context of the present application, BDDE is particularly preferred.

In one embodiment, the process for crosslinking a polymer according to the invention is characterized in that said at least one crosslinking agent is 1,4-butanediol diglycidyl ether (BDDE), and said step c) is carried out at a pH greater than 10.

In one embodiment, the process for crosslinking a polymer according to the invention is characterized in that said crosslinking step c) has a duration of between 3 and 72 hours.

In one embodiment, the process for crosslinking a polymer according to the invention is characterized in that said crosslinking step c) has a duration of between 3 and 60 hours.

In one embodiment, the process for crosslinking a polymer according to the invention is characterized in that said crosslinking step c) has a duration of between 3 and 50 hours.

In one embodiment, the process for crosslinking a polymer according to the invention is characterized in that said crosslinking step c) has a duration of between 5 and 50 hours.

In one embodiment, the process for crosslinking a polymer according to the invention is characterized in that said crosslinking step c) has a duration of between 10 and 50 hours.

In one embodiment, the process for crosslinking a polymer according to the invention is characterized in that said crosslinking step c) has a duration of between 15 and 48 hours.

In one embodiment, the process for crosslinking a polymer according to the invention is characterized in that said crosslinking step c) has a duration of between 20 and 30 hours.

In one embodiment, the process for crosslinking a polymer according to the invention is characterized in that said crosslinking step c) has a duration of between 21 and 28 hours.

When several successive crosslinkings are carried out, the durations mentioned are the total durations (the sum of the durations of the successive crosslinkings).

In one embodiment, during step c), the implementation of the crosslinking step(s) in the presence of said polymer and said crosslinking agent takes place in a reaction medium in which said polymer is hydrated and/or swollen.

In one embodiment, during step c), the implementation of the crosslinking step(s) in the presence of said polymer and said crosslinking agent takes place in a reaction medium in which said polymer is hydrated and/or swollen by adding water or an aqueous saline solution, for example a phosphate buffer solution, for example PBS. In one embodiment, during step c), the implementation of the crosslinking step(s) in the presence of said polymer and said crosslinking agent, the polymer concentration is between 0.05 and 30% by mass relative to the total mass of the crosslinking reaction medium.

In one embodiment, during step c), the implementation of the crosslinking step(s) in the presence of said polymer and said crosslinking agent, the polymer concentration is between 1 and 30% by mass relative to the total mass of the reaction medium.

In one embodiment, during step c), the implementation of the crosslinking step(s) in the presence of said polymer and said crosslinking agent, the polymer concentration is between 5 and 25% by mass relative to the total mass of the reaction medium.

In one embodiment, during step c), the implementation of the crosslinking step(s) in the presence of said polymer and said crosslinking agent, the polymer concentration is between 10 and 15% by mass relative to the total mass of the reaction medium.

In one embodiment, during step c), the implementation of the crosslinking step(s) in the presence of hyaluronic acid or any of its biologically acceptable salts, alone or in mixture, and said crosslinking agent, the hyaluronic acid concentration is between 0.05 and 30% by mass relative to the total mass of the reaction medium.

In one embodiment, during step c), the implementation of the crosslinking step(s) in the presence of hyaluronic acid or any of its biologically acceptable salts, alone or in mixture, and said crosslinking agent in a crosslinking reaction medium, the hyaluronic acid concentration is between 1 and 30% by mass relative to the total mass of the reaction medium.

In one embodiment, during step c), the implementation of the crosslinking step(s) in the presence of hyaluronic acid or any of its biologically acceptable salts, alone or in mixture, and said crosslinking agent, the polymer concentration is between 5 and 25% by mass relative to the total mass of the reaction medium.

In one embodiment, during step c), the implementation of the crosslinking step(s) in the presence of hyaluronic acid or any of its biologically acceptable salts, alone or in mixture, and said crosslinking agent, the hyaluronic acid concentration is between 10 and 15% by mass relative to the total mass of the reaction medium.

In one embodiment, during step c), the implementation of the crosslinking step(s) in the presence of said polymer and said crosslinking agent, the crosslinking reaction medium comprises sodium hydroxide (NaOH).

In one embodiment, during step c), the implementation of the crosslinking step(s) in the presence of said polymer and said crosslinking agent, the sodium hydroxide concentration is between 0.5 and 1.5% by mass relative to the total mass of the reaction medium.

In one embodiment, during step c), the implementation of the crosslinking step(s) in the presence of said polymer and said crosslinking agent, the sodium hydroxide concentration is between 0.8 and 1% by mass relative to the total mass of the reaction medium.

The invention also relates to a process for preparing a formulation comprising at least one crosslinked polymer obtained according to the process of the invention.

In one embodiment, the process for preparing a formulation comprising at least one crosslinked polymer obtained according to the process of the invention also comprises at least one step for hydration and/or swelling.

In one embodiment, the step for hydration and/or swelling in a liquid is performed by adding water or an aqueous saline solution, for example a phosphate buffer solution, for example PBS.

In one embodiment, the process for preparing a formulation comprising at least one crosslinked polymer obtained according to the process of the invention also comprises at least one step for hydration and/or swelling in an aqueous solution so as to obtain a polysaccharide concentration of between 2 mg/g and 50 mg/g relative to the total mass of said formulation.

In one embodiment, the process for preparing a formulation comprising at least one crosslinked polymer obtained according to the process of the invention also comprises at least one step for hydration and/or swelling in an aqueous solution so as to obtain a polysaccharide concentration of between 4 mg/g and 40 mg/g relative to the total mass of said formulation.

In one embodiment, the process for preparing a formulation comprising at least one crosslinked polymer obtained according to the process of the invention also comprises at least one step for hydration and/or swelling in an aqueous solution so as to obtain a polysaccharide concentration of between 5 mg/g and 30 mg/g relative to the total mass of said formulation.

In one embodiment, the process for preparing a formulation comprising at least one crosslinked polymer obtained according to the process of the invention also comprises at least one step for hydration and/or swelling in an aqueous solution so as to obtain a polysaccharide concentration of between 10 mg/g and 30 mg/g relative to the total mass of said formulation.

In one embodiment, the process for preparing a formulation comprising at least one crosslinked polymer obtained according to the process of the invention also comprises at least one step for hydration and/or swelling in an aqueous solution so as to obtain a polysaccharide concentration of around 20 mg/g relative to the total mass of said formulation.

In one embodiment, the process for preparing a formulation comprising at least one crosslinked polymer obtained according to the process of the invention comprises a step for the homogeneous mixing of Y identical or different crosslinked polymers, crosslinked prior to their interpenetration by mixing, Y being between 2 and 5, characterized in that at least one of the Y polymers is crosslinked according to the crosslinking process according to the invention.

In one embodiment, Y=2 and 1 polymer is crosslinked according to the process for preparing a crosslinked polymer according to the invention.

In one embodiment, Y=2 and 2 polymers are crosslinked according to the process for preparing a crosslinked polymer according to the invention.

In one embodiment, Y=3 and 1 polymer is crosslinked according to the process for preparing a crosslinked polymer according to the invention.

In one embodiment, Y=3 and 2 polymers are crosslinked according to the process for preparing a crosslinked polymer according to the invention.

In one embodiment, Y=3 and 3 polymers are crosslinked according to the process for preparing a crosslinked polymer according to the invention.

In one embodiment, Y=2, the 2 polymers are hyaluronic acids, or hyaluronic acid salts, having different molecular masses.

In one embodiment, the Y polymers are mixed prior to the swelling of each of said Y polymers.

In one embodiment, the Y polymers are mixed after the swelling of each of said Y polymers.

In one embodiment, the Y polymers are mixed prior to swelling. In one embodiment, the Y polymers are mixed after swelling.

In one embodiment, the process for preparing a formulation comprising at least one crosslinked polymer obtained according to the process of the invention also comprises at least one final sterilization step.

In one embodiment, the process for preparing the formulation comprising at least one crosslinked polymer according to the invention also comprises a final sterilization step.

In one embodiment, said final sterilization step is performed by heat, by moist heat, by gamma irradiation, by accelerated electron beam.

In one embodiment, said final sterilization step is performed by steam autoclaving.

In one embodiment, the steam autoclaving is performed with an F0≥4 minutes.

In one embodiment, the steam autoclaving is performed with an F0≥10 minutes.

In one embodiment, the steam autoclaving is performed with an F0≥15 minutes.

In one embodiment, the steam autoclaving is performed with an F0≥20 minutes.

In one embodiment, the process for preparing a formulation comprising at least one crosslinked polymer obtained according to the process of the invention also comprises at least one step for adding at least one active agent.

In one embodiment, the at least one active agent is added in powder form.

In one embodiment, the at least one active agent is added in solution or suspension form.

In one embodiment, the at least one active agent is added in solution or suspension form, in a solvent or solution chosen from the group comprised of water and aqueous saline solutions, for example a phosphate buffer solution, for example PBS.

In one embodiment, the process for preparing a formulation comprising at least one crosslinked polymer obtained according to the process of the invention also comprises at least one step for adding at least one active ingredient chosen from the group comprised of local anesthetics, vitamin C derivatives, anti-inflammatories, polyols, and their mixtures.

In one embodiment, the process for preparing a formulation comprising at least one crosslinked polymer obtained according to the process of the invention also comprises at least one step for adding at least one local anesthetic.

In one embodiment, the process for preparing a formulation comprising at least one crosslinked polymer obtained according to the process of the invention also comprises at least one step for adding at least one local anesthetic so as to obtain a local anesthetic concentration of between 0.1 and 5% relative to the total mass of said formulation.

In one embodiment, the process for preparing a formulation comprising at least one crosslinked polymer obtained according to the process of the invention also comprises at least one step for adding at least one local anesthetic so as to obtain a local anesthetic concentration of between 0.1 and 4% relative to the total mass of said formulation.

In one embodiment, the process for preparing a formulation comprising at least one crosslinked polymer obtained according to the process of the invention also comprises at least one step for adding at least one local anesthetic so as to obtain a local anesthetic concentration of between 0.1 and 2% relative to the total mass of said formulation.

In one embodiment, the process for preparing a formulation comprising at least one crosslinked polymer obtained according to the process of the invention also comprises at least one step for adding at least one local anesthetic so as to obtain a local anesthetic concentration of between 0.1 and 1% relative to the total mass of said formulation.

In one embodiment, the process for preparing a formulation comprising at least one crosslinked polymer obtained according to the process of the invention also comprises at least one step for adding at least one local anesthetic so as to obtain a local anesthetic concentration of between 0.1 and 0.5% relative to the total mass of said formulation.

In one embodiment, the process for preparing a formulation comprising at least one crosslinked polymer obtained according to the process of the invention also comprises at least one step for adding at least one local anesthetic so as to obtain a local anesthetic concentration of around 0.3% relative to the total mass of said formulation.

In one embodiment, the process for preparing a formulation comprising at least one crosslinked polymer obtained according to the process of the invention also comprises at least one step for adding at least one local anesthetic chosen from the group comprised of lidocaine, mepivacaine, and their mixtures.

In one embodiment, the process for preparing a formulation comprising at least one crosslinked polymer obtained according to the process of the invention also comprises at least one step for adding at least one local anesthetic, i.e. lidocaine.

In one embodiment, the process for preparing a formulation comprising at least one crosslinked polymer obtained according to the process of the invention also comprises at least one step for adding at least one local anesthetic, i.e. lidocaine, so as to obtain a lidocaine concentration of between 0.1 and 5% relative to the total mass of said formulation.

In one embodiment, the process for preparing a formulation comprising at least one crosslinked polymer obtained according to the process of the invention also comprises at least one step for adding at least one local anesthetic, i.e. lidocaine, so as to obtain a lidocaine concentration of between 0.1 and 4% relative to the total mass of said formulation.

In one embodiment, the process for preparing a formulation comprising at least one crosslinked polymer obtained according to the process of the invention also comprises at least one step for adding at least one local anesthetic, i.e. lidocaine, so as to obtain a lidocaine concentration of between 0.1 and 2% relative to the total mass of said formulation.

In one embodiment, the process for preparing a formulation comprising at least one crosslinked polymer obtained according to the process of the invention also comprises at least one step for adding at least one local anesthetic, i.e. lidocaine, so as to obtain a lidocaine concentration of between entre 0.1 and 1% relative to the total mass of said formulation.

In one embodiment, the process for preparing a formulation comprising at least one crosslinked polymer obtained according to the process of the invention also comprises at least one step for adding at least one local anesthetic, i.e. lidocaine so as to obtain a lidocaine concentration of between 0.1 and 0.5% relative to the total mass of said formulation.

In one embodiment, the process for preparing a formulation comprising at least one crosslinked polymer obtained according to the process of the invention also comprises at least one step for adding at least one local anesthetic, i.e. lidocaine, so as to obtain a local anesthetic concentration of around 0.3% relative to the total mass of said formulation.

In one embodiment, the process for preparing a formulation comprising at least one crosslinked polymer obtained according to the process of the invention also comprises at least one step for adding at least one local anesthetic, i.e. mepivacaine.

In one embodiment, the process for preparing a formulation comprising at least one crosslinked polymer obtained according to the process of the invention also comprises at least one step for adding at least one local anesthetic, i.e. mepivacaine, so as to obtain a mepivacaine concentration of between 0.1 and 5% relative to the total mass of said formulation.

In one embodiment, the process for preparing a formulation comprising at least one crosslinked polymer obtained according to the process of the invention also comprises at least one step for adding at least one local anesthetic, i.e. mepivacaine, so as to obtain a mepivacaine concentration of between 0.1 and 4% relative to the total mass of said formulation.

In one embodiment, the process for preparing a formulation comprising at least one crosslinked polymer obtained according to the process of the invention also comprises at least one step for adding at least one local anesthetic, i.e. mepivacaine, so as to obtain a mepivacaine concentration of between 0.1 and 2% relative to the total mass of said formulation.

In one embodiment, the process for preparing a formulation comprising at least one crosslinked polymer obtained according to the process of the invention also comprises at least one step for adding at least one local anesthetic, i.e. mepivacaine, so as to obtain a mepivacaine concentration of between 0.1 and 1% relative to the total mass of said formulation.

In one embodiment, the process for preparing a formulation comprising at least one crosslinked polymer obtained according to the process of the invention also comprises at least one step for adding at least one local anesthetic, i.e. mepivacaine, so as to obtain a mepivacaine concentration of between 0.1 and 0.5% relative to the total mass of said formulation.

In one embodiment, the process for preparing a formulation comprising at least one crosslinked polymer obtained according to the process of the invention also comprises at least one step for adding at least one local anesthetic, i.e. mepivacaine, so as to obtain a mepivacaine concentration of around 0.3% relative to the total mass of said formulation.

In one embodiment, the process for preparing a formulation comprising at least one crosslinked polymer obtained according to the process of the invention also comprises at least one step for adding at least one vitamin C derivative.

In one embodiment, the process for preparing a formulation comprising at least one crosslinked polymer obtained according to the process of the invention also comprises at least one step for adding at least one vitamin C derivative chosen from the group comprised of ascorbyl phosphates (such as for example magnesium ascorbyl phosphate, sodium ascorbyl phosphate), ascorbyl glycosides (such as for example ascorbic acid-2-glucoside), and their mixtures.

In one embodiment, said at least one vitamin C derivative is magnesium ascorbyl phosphate.

In one embodiment, the process for preparing a formulation comprising at least one crosslinked polymer obtained according to the process of the invention also comprises at least one step for adding at least one anti-inflammatory.

In one embodiment, the process for preparing a formulation comprising at least one crosslinked polymer obtained according to the process of the invention also comprises at least one step for adding at least one anti-inflammatory chosen from the group comprised of steroidal and nonsteroidal anti-inflammatories.

In one embodiment, said at least one anti-inflammatory is chosen from the group comprised of steroidal anti-inflammatories (such as for example dexamethasone, prednisolone, corticosterone, budesonide, sulfasalazine, mesalamine, cetirizine, diphenhydramine, antipyrine, methyl salicylate, loratadine, thymol, carvacrol, bisabolol, allantoin, eucalyptol, phenazone (antipyrine), propyphenazone) and nonsteroidal anti-inflammatories (such as for example ibuprofen, naproxen, fenoprofen, ketoprofen, flurbiprofen, oxaprozin, indometacin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, or sucrose octasulfate and/or its salts).

In one embodiment, said at least one anti-inflammatory is chosen from the group comprised of sucrose octasulfate and its salts.

In one embodiment, said at least one anti-inflammatory is chosen from the group comprised of sucrose octasulfate and its sodium and potassium salts.

In one embodiment, said water-soluble salt of sucrose octasulfate is chosen from the group comprised of alkali metal salts, alkaline earth metal salts, silver salts, ammonium salts, amino acid salts.

In one embodiment, said water-soluble salt of sucrose octasulfate is chosen from the group comprised of alkali metal salts or alkaline earth metal salts.

In one embodiment, said water-soluble salt of sucrose octasulfate is sucrose octasulfate sodium salt or sucrose octasulfate potassium salt.

In one embodiment, the process for preparing a formulation comprising at least one crosslinked polymer obtained according to the process of the invention also comprises at least one step for adding at least one polyol.

In one embodiment, the process for preparing a formulation comprising at least one crosslinked polymer obtained according to the process of the invention also comprises at least one step for adding at least one polyol chosen from the group comprised of mannitol, sorbitol, glycerol, maltitol, lactitol and erythritol.

In one embodiment, the process for preparing a formulation comprising at least one crosslinked polymer obtained according to the process of the invention also comprises at least one step for adding at least one polyol chosen from the group comprised of mannitol, sorbitol and glycerol.

In one embodiment, the process for preparing a formulation comprising at least one crosslinked polymer obtained according to the process of the invention also comprises at least one step for adding at least one polyol so as to obtain a polyol concentration of between 0.1 mg/ml and 50 mg/ml relative to the total mass of said formulation.

In one embodiment, the process for preparing a formulation comprising at least one crosslinked polymer obtained according to the process of the invention also comprises at least one step for adding at least one polyol so as to obtain a polyol concentration of between 5 mg/ml and 40 mg/ml relative to the total mass of said formulation.

In one embodiment, the process for preparing a formulation comprising at least one crosslinked polymer obtained according to the process of the invention also comprises at least one step for adding at least one polyol so as to obtain a polyol concentration of between 10 mg/ml and 40 mg/ml relative to the total mass of said formulation.

In one embodiment, the process for preparing a formulation comprising at least one crosslinked polymer obtained according to the process of the invention also comprises at least one step for adding at least one polyol so as to obtain a polyol concentration of between 20 mg/ml and 40 mg/ml relative to the total mass of said formulation.

In one embodiment, the process for preparing a formulation comprising at least one crosslinked polymer obtained according to the process of the invention also comprises at least one step for adding at least one polyol so as to obtain a polyol concentration of between 30 mg/ml and 40 mg/ml relative to the total mass of said formulation.

In one embodiment, the process for preparing a formulation comprising at least one crosslinked polymer obtained according to the process of the invention also comprises at least one step for adding at least one polyol, i.e. mannitol.

In one embodiment, the process for preparing a formulation comprising at least one crosslinked polymer obtained according to the process of the invention also comprises at least one step for adding mannitol so as to obtain a mannitol concentration of between 5 mg/ml and 40 mg/ml relative to the total mass of said formulation.

In one embodiment, the process for preparing a formulation comprising at least one crosslinked polymer obtained according to the process of the invention also comprises at least one step for adding mannitol so as to obtain a mannitol concentration of between 10 mg/ml and 40 mg/ml relative to the total mass of said formulation.

In one embodiment, the process for preparing a formulation comprising at least one crosslinked polymer obtained according to the process of the invention also comprises at least one step for adding mannitol so as to obtain a mannitol concentration of between 20 mg/ml and 40 mg/ml relative to the total mass of said formulation. In one embodiment, the process for preparing a formulation comprising at least one crosslinked polymer obtained according to the process of the invention also comprises at least one step for adding mannitol so as to obtain a mannitol concentration of between 30 mg/ml and 40 mg/ml relative to the total mass of said formulation.

In one embodiment, the process for preparing a formulation comprising at least one crosslinked polymer obtained according to the process of the invention also comprises at least one step for adding at least one polyol, i.e. sorbitol.

In one embodiment, the process for preparing a formulation comprising at least one crosslinked polymer obtained according to the process of the invention also comprises at least one step for adding sorbitol so as to obtain a sorbitol concentration of between 5 mg/ml and 40 mg/ml relative to the total mass of said formulation.

In one embodiment, the process for preparing a formulation comprising at least one crosslinked polymer obtained according to the process of the invention also comprises at least one step for adding sorbitol so as to obtain a sorbitol concentration of between 10 mg/ml and 40 mg/ml relative to the total mass of said formulation.

In one embodiment, the process for preparing a formulation comprising at least one crosslinked polymer obtained according to the process of the invention also comprises at least one step for adding sorbitol so as to obtain a sorbitol concentration of between 20 mg/ml and 40 mg/ml relative to the total mass of said formulation. In one embodiment, the process for preparing a formulation comprising at least one crosslinked polymer obtained according to the process of the invention also comprises at least one step for adding sorbitol so as to obtain a sorbitol concentration of between 30 mg/ml and 40 mg/ml relative to the total mass of said formulation.

The invention also relates to a formulation comprising at least one crosslinked polymer obtained according to the process of the invention.

In one embodiment, the formulation is characterized in that the polymer concentration is between 2 mg/g and 50 mg/g relative to the total mass of said formulation.

In one embodiment, the formulation is characterized in that the polymer concentration is between 4 mg/g and 40 mg/g relative to the total mass of said formulation.

In one embodiment, the formulation is characterized in that the polymer concentration is between 5 mg/g and 30 mg/g relative to the total mass of said formulation.

In one embodiment, the formulation is characterized in that the polymer concentration is between 10 mg/g and 30 mg/g relative to the total mass of said formulation.

In one embodiment, the formulation is characterized in that the polymer concentration is around 20 mg/g relative to the total mass of said formulation.

In one embodiment, the formulation is characterized in that it is injectable.

In one embodiment, the formulation is characterized in that it is sterile.

In one embodiment, the formulation is characterized in that it is monophasic.

In one embodiment, the formulation is characterized in that it is injectable and sterile.

In one embodiment, the formulation is characterized in that it is injectable, sterile and monophasic.

In one embodiment, said formulation comprising at least one polymer crosslinked according to the process of the invention comprises a homogeneous mixture of Y identical or different crosslinked polymers, crosslinked prior to their interpenetration by mixing, Y being between 2 and 5, characterized in that at least one of the Y polymers is crosslinked according to the process for preparing a crosslinked polymer according to the invention.

In one embodiment, Y=2 and 1 polymer is crosslinked according to the process for preparing a crosslinked polymer according to the invention.

In one embodiment, Y=2 and 2 polymers are crosslinked according to the process for preparing a crosslinked polymer according to the invention.

In one embodiment, Y=3 and 1 polymer is crosslinked according to the process for preparing a crosslinked polymer according to the invention.

In one embodiment, Y=3 and 2 polymers are crosslinked according to the process for preparing a crosslinked polymer according to the invention.

In one embodiment, Y=3 and 3 polymers are crosslinked according to the process for preparing a crosslinked polymer according to the invention.

In one embodiment, Y=2, the 2 polymers are hyaluronic acids, or hyaluronic acid salts, having different molecular masses.

In one embodiment, said formulation also comprises at least one active agent chosen from the group composed of local anesthetics, vitamin C derivatives, anti-inflammatories, polyols, and their mixtures.

In one embodiment, said formulation also comprises at least one local anesthetic.

In one embodiment, the mass percentage of said at least one local anesthetic is between 0.1 and 5% relative to the total mass of said formulation.

In one embodiment, the mass percentage of said at least one local anesthetic is between 0.1 and 4% relative to the total mass of said formulation.

In one embodiment, the mass percentage of said at least one local anesthetic is between 0.1 and 2% relative to the total mass of said formulation.

In one embodiment, the mass percentage of said at least one local anesthetic is between 0.1 and 1% relative to the total mass of said formulation.

In one embodiment, the mass percentage of said at least one local anesthetic is between 0.1 and 0.5% relative to the total mass of said formulation.

In one embodiment, the mass percentage of said at least one local anesthetic is around 0.3% relative to the total mass of said formulation.

In one embodiment, said formulation also comprises at least one active agent.

In one embodiment, said formulation also comprises at least one local anesthetic chosen from the group comprised of lidocaine, mepivacaine, and their mixtures.

In one embodiment, said local anesthetic is lidocaine.

In one embodiment, said local anesthetic is lidocaine, the mass percentage of being between 0.1 and 5% relative to the total mass of said formulation.

In one embodiment, said local anesthetic is lidocaine, the mass percentage of lidocaine being between 0.1 and 4% relative to the total mass of said formulation.

In one embodiment, said local anesthetic is lidocaine, the mass percentage of lidocaine being between 0.1 and 2% relative to the total mass of said formulation.

In one embodiment, said local anesthetic is lidocaine, the mass percentage of lidocaine being between 0.1 and 1% relative to the total mass of said formulation.

In one embodiment, said local anesthetic is lidocaine, the mass percentage of lidocaine being between 0.1 and 0.5% relative to the total mass of said formulation.

In one embodiment, said local anesthetic is mepivacaine.

In one embodiment, said local anesthetic is mepivacaine, the mass percentage of mepivacaine being between 0.1 and 5% relative to the total mass of said formulation.

In one embodiment, said local anesthetic is mepivacaine, the mass percentage of mepivacaine being between 0.1 and 4% relative to the total mass of said formulation.

In one embodiment, said local anesthetic is mepivacaine, the mass percentage of mepivacaine being between 0.1 and 2% relative to the total mass of said formulation.

In one embodiment, said local anesthetic is mepivacaine, the mass percentage of mepivacaine being between 0.1 and 1% relative to the total mass of said formulation.

In one embodiment, said local anesthetic is mepivacaine, the mass percentage of mepivacaine being between 0.1 and 0.5% relative to the total mass of said formulation.

In one embodiment, said formulation also comprises at least one vitamin C derivative.

In one embodiment, said at least one vitamin C derivative is chosen from the group comprised of ascorbyl phosphates (such as for example magnesium ascorbyl phosphate, sodium ascorbyl phosphate), ascorbyl glycosides (such as for example ascorbic acid-2-glucoside), and their mixtures.

In one embodiment, said at least one vitamin C derivative is magnesium ascorbyl phosphate.

In one embodiment, said formulation also comprises at least one anti-inflammatory.

In one embodiment, said at least one anti-inflammatory is chosen from the group comprised of steroidal and nonsteroidal anti-inflammatories.

In one embodiment, said at least one anti-inflammatory is chosen from the group comprised of steroidal anti-inflammatories (such as for example dexamethasone, prednisolone, corticosterone, budesonide, sulfasalazine, mesalamine, cetirizine, diphenhydramine, antipyrine, methyl salicylate, loratadine, thymol, carvacrol, bisabolol, allantoin, eucalyptol, phenazone (antipyrine), propyphenazone) and nonsteroidal anti-inflammatories (such as for example ibuprofen, naproxen, fenoprofen, ketoprofen, flurbiprofen, oxaprozin, indometacin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, or sucrose octasulfate and/or its salts).

In one embodiment, said at least one anti-inflammatory is chosen from the group comprised of sucrose octasulfate and its salts.

In one embodiment, said at least one anti-inflammatory is chosen from the group comprised of sucrose octasulfate and its sodium and potassium salts.

In one embodiment, said water-soluble salt of sucrose octasulfate is chosen from the group comprised of alkali metal salts, alkaline earth metal salts, silver salts, ammonium salts, amino acid salts.

In one embodiment, said water-soluble salt of sucrose octasulfate is chosen from the group comprised of alkali metal salts or alkaline earth metal salts.

In one embodiment, said water-soluble salt of sucrose octasulfate is sucrose octasulfate sodium salt or sucrose octasulfate potassium salt.

In one embodiment, said formulation also comprises at least one polyol.

In one embodiment, said formulation also comprises at least one polyol chosen from the group comprised of mannitol, sorbitol, glycerol, maltitol, lactitol and erythritol.

In one embodiment, said formulation also comprises at least one polyol chosen from the group comprised of mannitol, sorbitol and glycerol.

In one embodiment, said formulation further comprises at least mannitol.

In one embodiment, the mass percentage of said polyol being between 0.1 mg/ml and 50 mg/ml relative to the total mass of said formulation.

In one embodiment, the mass percentage of said polyol being between 5 mg/ml and 40 mg/ml relative to the total mass of said formulation.

In one embodiment, the mass percentage of said polyol being between 10 mg/ml and 40 mg/ml relative to the total mass of said formulation.

In one embodiment, the mass percentage of said polyol being between 20 mg/ml and 40 mg/ml relative to the total mass of said formulation.

In one embodiment, the mass percentage of said polyol being between 30 mg/ml and 40 mg/ml relative to the total mass of said formulation.

In one embodiment, said formulation further comprises at least mannitol, the mass percentage of said mannitol being between 0.1 mg/ml and 50 mg/ml relative to the total mass of said formulation.

In one embodiment, said formulation further comprises at least mannitol, the mass percentage of said mannitol being between 5 mg/ml and 40 mg/ml relative to the total mass of said formulation.

In one embodiment, said formulation further comprises at least mannitol, the mass percentage of said mannitol being between 10 mg/ml and 40 mg/ml relative to the total mass of said formulation.

In one embodiment, said formulation further comprises at least mannitol, the mass percentage of said mannitol being between 20 mg/ml and 40 mg/ml relative to the total mass of said formulation.

In one embodiment, said formulation further comprises at least mannitol, the mass percentage of said mannitol being between 30 mg/ml and 40 mg/ml relative to the total mass of said formulation.

The formulations obtained by the subject process of the invention have many applications.

The medical applications include, for example, injections for replacing deficient biological fluids, for example in the joints to replace synovial fluid, postsurgical injections for preventing peritoneal adhesions, periurethral injections for treating incontinence, and injections following presbyopia surgery. The esthetic applications include, for example, injections for filling wrinkles, fine lines and skin defects or for increasing volumes, for example of the lips, cheeks, etc.

The more particularly intended applications are the applications commonly associated with injectable viscoelastics and polysaccharides used or potentially usable in the following pathologies or treatments:
esthetic injections to the face: for filling wrinkles or skin defects, or volumizing (cheeks, chin, lips);
volumizing injections to the body: breast and buttocks augmentation, G spot augmentation, vaginoplasty, reconstruction of the vaginal labia, penis size augmentation;
in joint surgery and dental surgery, for example for filling periodontal pockets.
in arthritis treatment, injection into the joint to replace or increase deficient synovial fluid;
periurethral injection for treating urinary incontinence due to sphincter deficiency;
postsurgical injection, particularly for preventing peritoneal adhesions;
injection following presbyopia surgery by scleral laser incision;
injection into the vitreous cavity;
injection during cataract surgery;
injection for treating cases of vaginal dryness;
injection into the genital structures.

More particularly, in esthetic surgery, as a function of its viscoelastic and persistence properties, the formulations obtained by the subject process of the invention can be used:
for filling fine lines or medium or deep wrinkles, and be injected with needles of small diameter (27 gauge, for example);
as a volumizer, with injection by larger-diameter, from 22 to 26 gauge for example, and longer (30 to 40 mm for example) needles; in this case, its cohesive nature makes it possible to guarantee its retention at the injection site.

These exemplary uses are in no way limiting, and the formulations obtained according to the subject process of the invention are more generally provided for:
filling volumes;
generating spaces within certain tissues, thus promoting their optional functioning;
replacing deficient physiological fluids.

The following embodiments are applicable to the process for crosslinking a polymer according to the invention, to the process for preparing a formulation comprising a polymer obtained by the process according to the invention, to the formulation according to the invention, and to the various uses of said formulation.

In one embodiment, said polymer is chosen from the group of polysaccharides.

In one embodiment, said polymer is chosen from the group of glycosaminoglycans (GAG).

In one embodiment, said polymer is chosen from the group of glycosaminoglycans (GAG), such as for example chondroitin, keratan, heparin, heparosan, or hyaluronic acid, and their mixtures.

In one embodiment, said polymer is chosen from the group comprised of hyaluronic acid, keratan, heparin, cellulose, cellulose derivatives, alginic acid, xanthan, carrageenan, chitosan, chondroitin, heparosan, and their biologically acceptable salts, alone or in mixture.

In one embodiment, said polymer is hyaluronic acid or any of its biologically acceptable salts, alone or in mixture.

In the context of the present application, hyaluronic acid or any of its biologically acceptable salts, alone or in mixture, are preferred.

In one embodiment, said polymer is chosen from the group comprised of hyaluronic acid, sodium hyaluronate, and their mixtures.

In one embodiment, said polymer is hyaluronic acid.

In one embodiment, said polymer is chosen from the group comprised of sodium hyaluronate and potassium hyaluronate.

In one embodiment, said polymer is sodium hyaluronate.

In the context of the present application, sodium hyaluronate is the particularly preferred polymer.

In one embodiment, said polymer is a hyaluronic acid or one of its salts, chemically modified by substitution.

In one embodiment, said polymer is a hyaluronic acid or one of its salts, substituted by a group imparting lipophilic or hydrating properties, such as for example the hyaluronic acids substituted as described in the patent application FR 2 983 483 in the name of the applicant.

In the context of the present application, Mw or “molecular mass” means the average molecular mass by weight of the polymers, measured in Daltons.

In one embodiment, said hyaluronic acid or one of its salts has a molecular mass between 0.01 MDa and 5 MDa.

In one embodiment, said hyaluronic acid or one of its salts has a molecular mass between 0.01 MDa and 3.5 MDa.

In one embodiment, said hyaluronic acid or one of its salts has a molecular mass between 0.5 MDa and 3.5 MDa.

In one embodiment, said hyaluronic acid or one of its salts has a molecular mass between 2.75 MDa and 3.25 MDa.

In one embodiment, said hyaluronic acid or one of its salts has a molecular mass between 0.75 MDa and 1.25 MDa.

In one embodiment, said hyaluronic acid or one of its salts has a molecular mass between 2 MDa and 5 MDa.

In one embodiment, said hyaluronic acid or one of its salts has a molecular mass between 2 MDa and 4 MDa.

In one embodiment, said hyaluronic acid or one of its salts has a molecular mass between 0.5 MDa and 2 MDa.

In one embodiment, said hyaluronic acid or one of its salts has a molecular mass between 0.5 MDa and 1.5 MDa.

In one embodiment, said at least one crosslinking agent is chosen from the group comprised of ethylene glycol diglycidyl ether, butanediol diglycidyl ether (BDDE), polyglycerol polyglycidyl ether, polyethylene glycol diglycidyl ether, polypropylene glycol diglycidyl ether, a bis- or polyepoxide such as 1,2,3,4-diepoxybutane or 1,2,7,8-diepoxyoctane, a dialkyl sulfone, divinyl sulfone, formaldehyde, epichlorohydrin or glutaraldehyde, carbodiimides such as for example 1-ethyl-3-[3-dimethylaminopropy]carbodiimide hydrochloride (EDC), trimetaphosphates, such as for example sodium trimetaphosphate, calcium trimetaphosphate, or barium trimetaphosphate.

In one embodiment, said at least one crosslinking agent is chosen from the group comprised of ethylene glycol diglycidyl ether, butanediol diglycidyl ether (BDDE), polyglycerol polyglycidyl ether, polyethylene glycol diglycidyl ether, polypropylene glycol diglycidyl ether, a bis- or polyepoxide such as 1,2,3,4-diepoxybutane or 1,2,7,8-diepoxyoctane, trimetaphosphates such as for example sodium trimetaphosphate, calcium trimetaphosphate, or barium trimetaphosphate.

In one embodiment, said at least one crosslinking agent is chosen from the group comprised of ethylene glycol diglycidyl ether, butanediol diglycidyl ether (BDDE), polyglycerol polyglycidyl ether, polyethylene glycol diglycidyl ether, polypropylene glycol diglycidyl ether, a bis- or polyepoxide such as 1,2,3,4-diepoxybutane or 1,2,7,8-diepoxyoctane.

In one embodiment, said at least one crosslinking agent is chosen from the group comprised of trimetaphosphates such as for example sodium trimetaphosphate, calcium trimetaphosphate, or barium trimetaphosphate.

In one embodiment, said at least one crosslinking agent is chosen from the group comprised of epoxides, for example 1,4-butanediol diglycidyl ether (BDDE), epihalohydrins, and divinyl sulfone (DVS).

In one embodiment, said at least one crosslinking agent is divinyl sulfone (DVS).

In one embodiment, said at least one crosslinking agent is 1,4-butanediol diglycidyl ether (BDDE).

In the context of the present application, BDDE is particularly preferred.

In one embodiment, the crosslinking rate X is between 0.001 and 0.5.

In one embodiment, the crosslinking rate X is between 0.01 and 0.4.

In one embodiment, the crosslinking rate X is between 0.03 and 0.23.

In one embodiment, the crosslinking rate X is between 0.03 and 0.20.

In one embodiment, the crosslinking rate X is between 0.03 and 0.15.

In one embodiment, the crosslinking rate X is between 0.03 and 0.10.

In one embodiment, the crosslinking rate X is between 0.10 and 0.15.

In one embodiment, the crosslinking rate X is between 0.08 and 0.15.

In one embodiment, the degree of modification of said crosslinked polymer is less than 5%.

In one embodiment, the degree of modification of said crosslinked polymer is less than 4%.

In one embodiment, the degree of modification of said crosslinked polymer is less than 3.5%.

In one embodiment, the degree of modification of said crosslinked polymer is less than 3%.

In one embodiment, the degree of modification of said crosslinked polymer is less than 2.5%.

### EXAMPLES

In the context of the examples, a certain number of parameters were measured.

### Determination of the Rheological Parameters G′, G″ and Tan Δ (Tn δ):

TA Instruments DHR-2 equipment. Cone type geometry with an angle of 2° and a diameter of 40 mm. Frequency sweep method (logarithmic sweep), deformation (strain) of 0.8% (deformation in the linear range), frequency range of 0.08 to 5 Hz, values read at the frequency of 1 Hz.

### Determination of the MoD:

The proton NMR spectra are obtained in a 400 MHz spectrometer. The MoD value is calculated from the integrals of the N-acetyl signal group of the hyaluronic acid and a BDDE signal (two —CH₂— groups). The ratio of the integrals of these two signals (crosslinking agent/NAc HA) corresponds to the MoD.

The value of the Mod (%) was determined using the process mentioned above (hyaluronic acid crosslinked by BDDE), and using the formula also mentioned above.

### Injectability Measurement:

A traction bench and a force gauge (N) are used. The traction bench applies a displacement speed to the plunger rod of a syringe and the gel is expelled from the needle (27 G1/2); the force in Newtons is recorded by the gauge during the ejection of the gel at a speed 13 mm/min.

### Evaluation of the Enzyme Degradation Resistance (Hyaluronidases):

A hyaluronidase solution (Sigma Aldrich H3506) (see Table 7 for the U/g value in a phosphate buffer) is prepared. This solution (20 μL) is mixed with 1 g of the gel to test, and everything is maintained at 37° C. for 5 to 10 minutes.

The gel mixed with the enzymes is then rheologically analyzed, TA Instruments DHR-2 equipment. Geometry with an angle of 2° and a diameter of 40 mm. Frequency oscillation method (logarithmic sweep), deformation (strain) of 0.8%, temperature of 37° C., fixed frequency of 1 Hz applied.

The analysis consists of monitoring the loss of G′ (Pa) as a function of time. The time at which the initial G′ of the formulation is reduced by half corresponds to the half-life of the analyzed product.

### Measurement of the Plastic Range, Rheological Deformation Measurements:

TA Instruments DHR-2 equipment. Geometry with an angle of 2° and a diameter of 40 mm. Strain sweep method: logarithmic sweep, deformation (strain) of 0.1 to 1000%, frequency of 1 Hz.

Observation of the plastic range, running from the deformation (in %) at which the G′ (Pa) drops by 10% relative to the initial G′ to the deformation (in %) at the crossover of the G′ and the G″.

### Example 1: Rheological Properties of a Formulation Obtained According to the Process of the Invention

Example 1 illustrates the influence of the implementation of the process according to the invention on the properties (G′, G″ and Tan Δ (Tn δ), Mod) of the formulation obtained. In this example, the properties (G′, G″, Tan Δ (Tn δ) and Mod) of a formulation obtained according to the process of the invention were compared to those of a formulation obtained by means of a conventionally used crosslinking (of the described in the application WO2009071697).

### Processes for Preparing the Formulations

The two compared formulations are each prepared according to the following process:

Sodium hyaluronate fibers of injectable quality (1 g; molecular mass: 3 MDa) are weighed in a vessel. A 1% aqueous solution of sodium hydroxide in water (7.4 g) is added, and everything is homogenized with a spatula for about 1 hour at ambient temperature and at 900 mm Hg.

An appropriate quantity of BDDE for obtaining a crosslinking rate X of around 0.14 is added to the non-crosslinked sodium hyaluronate gel obtained in the preceding step, and everything is homogenized with a spatula for about 30 minutes at ambient temperature and at 900 mm Hg.

The crosslinking conditions are as follows:
3 h 10 m at 50° C. for the (comparative) process of the prior art; and
23 to 26 h at around 9° C. for the process of the invention.

For each of the processes, the final crosslinked gel is then neutralized by adding HCl 1N and placed in a phosphate buffer bath in order to stabilize the pH and enable it to be hydrated or swollen to a hyaluronic acid concentration of 30 mg/g. The gel is then dialyzed in a phosphate buffer bath until a hyaluronic acid concentration of 20.9 mg/g is obtained. The pH of the gels at the end of this step corresponds to the pH of the buffer, or around 7.2. The final gels are then homogenized, and a measurement of the parameters (G′, G″, Mod) is performed.

In summary, the two compared processes differ only by the crosslinking temperature and crosslinking time conditions.

Properties of the Formulations Obtained (Before Sterilization)

The results of the determination of the rheological parameters and the Mod are given in Table 1 below:

TABLE 1
Example 1 - Properties of the formulations before sterilization

| | Conventionally | Process according |
|---|---|---|
| | used process | to the invention |
| Crosslinking conditions | 3 h 10 m - 50° C. | 23 to 26 h - around 9° C. |
| Hyaluronic acid | 3 MDa | 3 MDa |
| G′ (Pa) 1 Hz of the | 253 | 401 |
| formulation obtained | | |
| G″ (Pa) 1 Hz of the | 33 | 107 |
| formulation obtained | | |
| Tan Δ (tan δ) 1 Hz of the | 0.13 | 0.27 |
| formulation obtained | | |
| Mod as a % of the | 5.9 | 3.4 |
| formulation obtained | | |

It is noted that the formulations obtained by means of the process according to the invention have a G′ (401 Pa) much higher than that of the compositions obtained according to the process of the prior art (253 Pa).

Also, the value of G″ is more than three times higher with the process according to the present invention compared to the conventionally used process.

Lastly, quite surprisingly, these improved rheological properties are obtained even though the Mod as a % of the formulation prepared by means of the process according to the invention is lower.

Properties of the Formulations Obtained (after Sterilization)

The two formulations are sterilized by autoclaving (F0=44 min), and a second measurement of the same parameters (G′, G″) is performed.

The results of the determination of the rheological parameters and the Mod are given in Table 2 below:

TABLE 2
example 1 - Properties of the formulations after sterilization

| | Conventionally | Process according |
|---|---|---|
| | used process | to the invention |
| Crosslinking conditions | 3 h 10 m - 50° C. | 23 à 26 h - around 9° C. |
| Hyaluronic acid | 3 MDa | 3 MDa |
| Sterilization conditions | F0 = 44 | F0 = 44 |
| (autoclaving) F0 in min | | |
| G′ (Pa) 1 Hz of the | 159 | 153 |
| formulation obtained | | |
| (after sterilization) | | |
| G″ (Pa) 1 Hz of the | 27 | 80 |
| formulation obtained | | |
| (after sterilization) | | |
| Tan Δ (tan δ) 1 Hz of the | 0.17 | 0.52 |
| formulation obtained | | |
| (after sterilization) | | |
| pH (after sterilization) | 7.2 | 7.3 |

It is noted that the G′ of the formulation prepared with the process according to the invention (401 Pa/153 Pa) is more affected by the sterilization than the G′ of the formulation prepared with the conventionally used process (253 Pa/159 Pa), the G′ values of the two formulations being similar after sterilization.

It is noted that the G″ of the formulation prepared with the process according to the invention remains much higher than the G″ of the formulation prepared with the conventionally used process after sterilization.

It follows from the above that the Tan Δ (tan δ) of the formulation prepared with the process according to the invention is doubled during sterilization (0.27/0.52), while the Tan Δ (tan δ) value of the formulation prepared with the conventionally used process is modified relatively little (0.13/0.17).

In summary, the Tan Δ value, already higher for the formulation prepared according to the invention before sterilization, is further increased during the sterilization step.

The process according to the invention therefore makes it possible to obtain formulations having very good rheological properties while retaining a relatively low Mod (%) (good crosslinking efficiency). In our example, the gel has an equivalent rigidity G′ and reveals a deformation (optimized damping), the gel is characterized as less brittle.

### Injectability of the Formulation Prepared According to the Process of the Invention

The Injectability of the formulation prepared according to the process of the invention was measured.

After sterilization, an injection force of less than 35 N at 13 mm/min is observed for the gel obtained by the conventionally used process and by the process according to the invention. This makes it suitable for the applications envisioned in this application.

The formulation according to the invention is therefore a formulation that can be qualified as injectable.

### Example 2: Rheological Properties of a Formulation Obtained According to the Subject Process of the Invention

The processes used in Example 2 are identical to those of Example 1, except for the fact that the two formulations are based on hyaluronic acid with an average molecular mass by weight of 0.9 MDa and have a crosslinking rate X approximately equal to 0.09.

The results of the determination of the rheological parameters and the Mod are given in Table 3 below:

TABLE 3
Example 2 - Properties of the formulations before sterilization

| | Conventionally | Process according |
|---|---|---|
| | used process | to the invention |
| Crosslinking conditions | 3 h 10 m - 50° C. | 23 a 26 h - around |
| | | 9° C. |
| Hyaluronic acid | 0.9 MDa | 0.9 MDa |
| G′ (Pa) 1 Hz of the | 432 | 198 |
| formulation obtained | | |
| G″ (Pa) 1 Hz of the | 46 | 89 |
| formulation obtained | | |
| Tan Δ (tan δ) 1 Hz of the | 0.11 | 0.45 |
| formulation obtained | | |
| Mod as a % of the | 4.5 | 2.4 |
| formulation obtained | | |

It is noted that the formulation obtained by means of the process according to the invention has a G′ lower than that of the formulation obtained according to the process of the prior art.

The G″ value of the formulation obtained by means of the subject process of the invention is two times greater than that of the formulation obtained according to the conventionally used process.

It follows from the above that the value of Tan Δ 1 Hz is optimized in the formulation obtained according to the process of the invention.

Here again, the formulation obtained by the subject process of the invention has improved rheological properties while also having a relatively low Mod (%).

### Example 3: Rheological Properties of a Formulation Obtained According to the Subject Process of the Invention

The two formulations of Examples 1 and 2 prepared according to the conventionally used processes (before sterilization) are mixed in 50/50 proportions. This results in a formulation comprising two pre-crosslinked and mixed/interpenetrated formulations.

The two formulations of Examples 1 and 2 prepared according to the processes according to the invention (before sterilization) are also mixed in 50/50 proportions. This results in a formulation comprising two pre-crosslinked and mixed/interpenetrated formulations.

The results of the determination of the rheological parameters and the Mod are given in Table 4 below:

TABLE 4
Example 3 - Interpenetrated formulations

| | Conventionally | Interpenetrated |
|---|---|---|
| | used interpenetrated | formulation according |
| | formulation | to the invention |
| G′ (Pa) 1 Hz of the | 374 | 358 |
| formulation obtained | | |
| G″ (Pa) 1 Hz of the | 42 | 119 |
| formulation obtained | | |
| Tan Δ (tan δ) 1 Hz of the | 0.11 | 0.33 |
| formulation obtained | | |
| Mod as a % of the | 5.2 | 2.7 |
| formulation obtained | | |

It is noted that the G′ value (358 Pa) of the interpenetrated formulation according to the invention is much closer to the high value (Example 1; 401 Pa) than to the low value (Example 2; 198 Pa) of the formulations that constitute it.

It is also noted that the G″ value (119 Pa) of the formulation according to the invention is higher than each of the values of the formulations that constitute it (Example 1, 107 Pa; Example 2, 89 Pa).

In summary, the rheological properties of the interpenetrated formulations prepared according to the process of the invention have particularly surprising and unexpected rheological characteristics.

The formulation obtained according to the process according to the invention was then sterilized (F0=14.5 minutes) and tested for injectability.

It was demonstrated that at a speed of 13 mm/min and at ambient temperature (Gauge 27^{1/2}), the injectability is less than 35 N. The formulation obtained according to the process according to the invention is therefore perfectly injectable.

### Example 4: Use of the Invention on Hyaluronic Acids with High Masses

Sodium hyaluronate fibers of injectable quality (1 g; molecular mass: 3 MDa) are weighed in a vessel. A 1% aqueous solution of sodium hydroxide in water (7.4 g) is added, and everything is homogenized with a spatula for about 1 hour at ambient temperature and at 900 mm Hg.

An appropriate quantity of BDDE for obtaining a crosslinking rate X of around 0.14 is added to the non-crosslinked sodium hyaluronate gel obtained in the preceding step, everything being homogenized with a spatula for about 30 minutes at ambient temperature and at 900 mm Hg.

The crosslinking conditions of the four tests are as follows:
3 h 10 m at 50° C. for the (comparative) process of the prior art,
24 h at 9° C.,
24 h at 2° C.,
48 h at 9° C. for the process according to the invention.

For each of the processes, the final crosslinked gel is then neutralized by adding HCl 1N, and placed in a phosphate buffer bath in order to stabilize the pH and enable it to be hydrated or swollen to a hyaluronic acid concentration of about 40 mg/g. The gel is then dialyzed in a phosphate buffer bath until a hyaluronic acid concentration of about 26 mg/g is obtained. The pH of the gels at the end of this step corresponds to the pH of the buffer, or about 7.2. The final gels are subsequently homogenized, then sterilized in the autoclave, and the following measurements are performed:
G′, G″; for all of the reaction times and temperatures.
MoD; for the reaction times of 24 and 48 h and the temperature of 9° C.
Half-life (resistance to hyaluronidase enzymes); for the reaction time of 48 h and the temperature of 9° C.

TABLE 5
Example 4 - Rheological properties of
the formulations after sterilization

| | Conventionally | | | |
|---|---|---|---|---|
| | used process | Process according to the invention | | |
| Crosslinking | 3 h 10 m - | 24 h - | 24 h - | 48 h - |
| conditions | 50° C. | 9° C. | 2° C. | 9° C. |
| Hyaluronic acid | 3 MDa | 3 MDa | 3 MDa | 3 MDa |
| G′ (Pa) 1 Hz of | 208 | 257 | 162 | 309 |
| the formulation | | | | |
| obtained | | | | |
| G″ (Pa) 1 Hz of | 46 | 132 | 122 | 110 |
| the formulation | | | | |
| obtained | | | | |
| Tan Δ (Tn δ) 1 | 0.22 | 0.51 | 0.75 | 0.36 |
| Hz of the | | | | |
| formulation | | | | |
| obtained | | | | |

It is noted that the G′ values of the formulas at the reaction time of 24 h are relatively close to the reference formula while the Tan Δ (Tn δ) is optimized.

We observe that the more the reaction time and temperature increase (according to the invention), the closer the Tan Δ (Tn δ) approaches that of the conventionally used process.

TABLE 6
Example 4 - MoD of the formulations

| | Conventionally | | |
|---|---|---|---|
| | used process | Process according to the invention | |
| Crosslinking | 3 h 10 m - | 24 h - | 48 h - |
| conditions | 50° C. | 9° C. | 9° C. |
| Hyaluronic acid | 3 MDa | 3 MDa | 3 MDa |
| MoD as a % of | 5.8 | 3.1 | 4.7 |
| the formulation | | | |
| obtained | | | |

Surprisingly, the 48 h-9° C. process makes it possible to simultaneously obtain an optimized G′ and Tan Δ (Tn δ) and reduce the MoD (%). The formula therefore imparts properties that are optimized for a filling application (a formula both more rigid and having a better damping capacity), with improved biocompatibility.

TABLE 7
Example 4 - Enzymatic resistances
(persistence) of the formulations

| | Conventionally | Process according |
|---|---|---|
| | used process | to the invention |
| Crosslinking conditions | 3 h 10 m - 50° C. | 48 h - 9° C. |
| Hyaluronic acid | 3 MDa | 3 MDa |
| Half-life (min) | 18.2 | 19.4 |
| Concentration of the | 1750 | 2500 |
| hyaluronidase solution | | |
| placed in contact with | | |
| the gel, in U/g | | |

The results of the above table are unexpected in that, the MOD of the 48 h-9° C. formulation being lower than the reference (conventionally used process), the hyaluronic acid has fewer crosslinking bridges and should have a lower persistence.

Surprisingly, the measurements show the opposite; the half-lives are in fact similar, but with a higher enzyme concentration for the invention.

In summary, the formula crosslinked for 48 h at 9° C. has significant and surprising advantages such as good deformation capacity, retained rigidity, as well as optimized biocompatibility and resistance to enzymatic degradation.

### Example 5: Use of the Invention on Hyaluronic Acids with Average Masses

The processes used in this example are identical to those of Example 4, except for the fact that the two formulations are based on hyaluronic acid with an average molecular mass by weight of 0.9 MDa and have a crosslinking rate X approximately equal to 0.09.

The crosslinking conditions for the four tests are as follows:
3 h 10 m at 50° C. for the (comparative) process of the prior art,
24 h at 9° C.,
24 h at 2° C.,
48 h at 9° C. for the process according to the invention.

For each of the processes, the final crosslinked gel is then neutralized by adding HCl 1N and placed in a phosphate buffer bath in order to stabilize the pH and enable it to be hydrated or swollen to a hyaluronic acid concentration of about 40 mg/g. The gel is then dialyzed in a phosphate buffer bath until a hyaluronic acid concentration of about 26 mg/g is obtained. The pH of the gels at the end of this step corresponds to the pH of the buffer, or about 7.2. The final gels are then homogenized and analyzed for G′/G″ for all the reaction times. The measurements of the MoD are also shown for the reaction times of 24 and 48 h and the temperature of 9° C.

The formulations are then sterilized in the autoclave and the measurements of G′/G″ for all the reaction times are performed again.

TABLE 8
Example 5 - Rheological properties of
the formulations before sterilization

| | Conventionally | | |
|---|---|---|---|
| | used process | Process according to the invention | |
| Crosslinking | 3 h 10 m - | 24 h - | 24 h - |
| conditions | 50° C. | 9° C. | 2° C. |
| Hyaluronic acid | 0.9 MDa | 0.9 MDa | 0.9 MDa |
| G′ (Pa) 1 Hz of | 557 | 397 | 285 |
| the formulation | | | |
| obtained | | | |
| G″ (Pa) 1 Hz of | 55 | 140 | 152 |
| the formulation | | | |
| obtained | | | |
| Tan Δ (Tn δ) 1 | 0.10 | 0.35 | 0.53 |
| Hz of the | | | |
| formulation | | | |
| obtained | | | |

Before sterilization, we observe here again an optimization of the G″ and thus of the Tan Δ (Tn δ) as a result of the invention.

The strain sweep represented in FIG. 2 (Strain sweep curves) also surprisingly demonstrates a highly optimized plastic range for the 48 h-9° C. formula. It is noted on this curve that at equivalent G′, the product obtained by the process according to the invention has the widest plastic range.

TABLE 9
Example 5 - MoD of the formulations

| | Conventionally | | |
|---|---|---|---|
| | used process | Process according to the invention | |
| Crosslinking | 3 h 10 m - | 24 h - | 48 h - |
| conditions | 50° C. | 9° C. | 9° C. |
| Hyaluronic acid | 0.9 MDa | 0.9 MDa | 0.9 MDa |
| MoD as a % of the | 4.5 | 2.4 | 3.1 |
| formulation | | | |
| obtained | | | |

The MoD values obtained are relatively low. Surprisingly, it is observed that the G′ of the reference and of the 48 h-9° C. process are identical, while the MoD (%) of the invention is lower. The product obtained has the same rigidity performance with a minimal transformation of the hyaluronic acid.

TABLE 10
Example 5 - Rheological properties of
the formulations after sterilization

| | Conventionally | | |
|---|---|---|---|
| | used process | Process according to the invention | |
| Crosslinking | 3 h 10 m - | 24 h - | 48 h - |
| conditions | 50° C. | 9° C. | 9° C. |
| Hyaluronic acid | 0.9 MDa | 0.9 MDa | 0.9 MDa |
| G′ (Pa) 1 Hz of | 394 | 142 | 288 |
| the formulation | | | |
| obtained | | | |
| G″ (Pa) 1 Hz of | 46 | 88 | 85 |
| the formulation | | | |
| obtained | | | |
| Tan Δ (Tn δ) 1 | 0.12 | 0.62 | 0.30 |
| Hz of the | | | |
| formulation | | | |
| obtained | | | |

Here again, the 48 h-9° C. formula is very advantageous, and makes it possible with a relatively unchanged G′ to significantly improve the deformability of the product.

1. A process for crosslinking a polymer, comprising at least the following steps:
a) providing a polymer;
b) providing a crosslinking agent;
c) carrying out one or more crosslinking step(s) in the presence of said polymer and said crosslinking agent;
d) obtaining a crosslinked polymer;
wherein the crosslinking step or each of the crosslinking steps is carried out at a constant temperature or at a temperature that varies linearly or in a stepwise manner, said constant or variable temperature being less than or equal to 15° C., and in that said crosslinking step c) has a duration of between 3 and 72 hours.

2. The process for crosslinking a polymer according to claim 1, wherein the crosslinking step or each of the crosslinking steps is carried out at a constant temperature.

3. The process for crosslinking a polymer according to claim 1, wherein the crosslinked polymer obtained in step d) has Tan Δ (Tn δ)≥0.25.

4. The process for crosslinking a polymer according to claim 1, wherein the crosslinked polymer obtained in step d) has a G′≤1000.

5. The process for crosslinking a polymer according to claim 1, wherein said polymer is chosen from the group of polysaccharides.

6. A process for preparing a formulation comprising at least one crosslinked polymer obtained according to the process of claim 1.

7. The process according to claim 5, said formulation comprising at least one polymer crosslinked according to the process comprises a homogeneous mixture of Y identical or different crosslinked polymers, crosslinked prior to their interpenetration by mixing, Y being between 2 and 5, wherein at least one of the Y polymers is crosslinked according to the process for preparing a crosslinked polymer.

8. The process according to claim 5, wherein said polymer is chosen from the group of polysaccharides.

9. A formulation comprising at least one crosslinked polymer obtained according to the process of claim 1.

10. The formulation according to claim 9, wherein it also comprises at least one active agent chosen from the group composed of local anesthetics, vitamin C derivatives, anti-inflammatories, polyols, and their mixtures.

11. A polymer wherein it has a G′≤1000 Pa and a tan Δ (Tn δ) whose value is between 0.25 and 1 (0.25≤Tan Δ (Tn δ))≤1).

12. The polymer according to claim 11, wherein it is chosen from the group of polysaccharides.

13. The polymer according to claim 11 wherein it is comprised of a mixture of hyaluronic acids, or of hyaluronic acid salts.
